(19) United States
(12) Patent Application Publication
(10) Pub. No.: US 2022/0101998 A1
(43) Pub. Date: Mar. 31, 2022

(54) TUMOR RECURRENCE PREDICTION DEVICE AND METHOD

(71) Applicants: Taipei Medical University, Taipei City (TW); TAIPEI VETERANS GENERAL HOSPITAL, Taipei City (TW)

(72) Inventors: Syu-Jyun PENG, Zhubei City (TW); Cheng-Chia LEE, Taipei City (TW); Huai-Che YANG, Taipei City (TW); Jing-Yu YANG, Taipei City (TW); Chih-Ying HUANG, Kaohsiung City (TW); Yi-Chen CHEN, Taipei City (TW); Hsiu-Mei WU, Taipei City (TW)

(21) Appl. No.: 17/187,466

(22) Filed: Feb. 26, 2021

(30) Foreign Application Priority Data
Sep. 28, 2020 (TW) 109133731

(51) Int. Cl. G16H 50/20; G06T 7/00; G06T 7/70; G16H 10/60; G16H 30/40; G06N 20/00

(52) U.S. Cl. CPC G16H 50/20; G06T 7/0012; G06T 7/70; G16H 10/60; G16H 30/40; G06T 2207/10088; G06T 2207/20081; G06T 2207/30096; G06T 2207/20084; G06T 2207/30016; G06N 20/00

(57) ABSTRACT

A tumor recurrence prediction device is provided, which includes a data extraction circuit, a memory, and a processor. The data extraction circuit extracts multiple patient clinical data and multiple slice image information; a memory stores multiple instructions; a processor is connected to the data extraction circuit and the memory, and is configured to load and execute the multiple instructions to: receive the multiple patient clinical data and the multiple slice image information; generate clinical feature information and tumor image feature information according to the multiple patient clinical data and the multiple slice image information; train a prediction model according to the clinical feature information and the tumor image feature information; and predict tumor recurrence for patient information of a patient using the prediction model. In addition, a tumor recurrence prediction method is also disclosed here.

### CROSS-REFERENCE TO RELATED APPLICATION

This application claims priority to Taiwan Application Serial Number 109133731, filed Sep. 28, 2020, which is herein incorporated by reference in its entirety.

### BACKGROUND

### Field of Disclosure

The present disclosure relates to a tumor recurrence prediction device and method. More particularly, the present disclosure relates to a tumor recurrence prediction device and method that improve the prediction accuracy of patient tumor recurrence.

### Description of Related Art

In hospitals or hospital systems, brain metastases are the most common malignant intracranial tumors, and the most common primary lesion is lung cancer. The Non-Small cell lung cancer (NSCLC) accounts for about 80% of all lung cancers and 25-50% of patients with metastatic NSCLC are affected by brain metastases during the course of their disease. Despite advances in the current systemic therapy and improvement of survival rates for patients with advanced NSCLC, the brain metastases are still the main cause of morbidity and death. Therefore, how to predict whether the brain metastasis tumor recurrence or the time of recurrence is an urgent problem for those skilled in the art.

### SUMMARY

The disclosure provides a tumor recurrence prediction device. The tumor recurrence prediction device comprises a data extraction circuit, a memory, and a processor. The data extraction circuit extracts a plurality of patient clinical data and a plurality of slice image information; a memory stores a plurality of instructions; a processor is connected to the data extraction circuit and the memory, and is configured to load and execute the plurality of instructions to: receive the plurality of patient clinical data and the plurality of slice image information; generate clinical feature information and tumor image feature information according to the plurality of patient clinical data and the plurality of slice image information; train a prediction model according to the clinical feature information and the tumor image feature information; and predict tumor recurrence for patient information of a patient using the prediction model.

The disclosure provides a tumor recurrence method. The tumor recurrence prediction method comprises: generating patient feature information and tumor image feature information according to a plurality of patient clinical data and a plurality of slice image information; combining the clinical feature information with the tumor image feature information to generate a feature array, and training a prediction model according to the feature array; and predicting tumor recurrence for patient information of a patient using the prediction model.

Based on the above, the tumor recurrence prediction device of the present disclosure combines the feature extraction of multiple patient clinical data and multiple tumor image information, and trains a prediction model using the extracted feature information to solve the current problem of poor accuracy of survival prediction analysis.

It is to be understood that both the foregoing general description and the following detailed description are by examples, and are intended to provide further explanation of the disclosure as claimed.

### BRIEF DESCRIPTION OF THE DRAWINGS

The disclosure can be more fully understood by reading the following detailed description of the embodiment, with reference made to the accompanying drawings as follows:

FIG. 1 is a block diagram of a tumor recurrence prediction device according to an embodiment of the present disclosure,

FIG. 2 is a flowchart of a tumor recurrence prediction method according to an embodiment of the present disclosure,

FIG. 3 is a schematic diagram of the tumor recurrence prediction method according to an embodiment of the present disclosure,

FIG. 4 is a schematic diagram of performing image processing on slice image information according to an embodiment of the present disclosure,

FIG. 5 is a schematic diagram of a tumor image corresponding to tumor location information according to an embodiment of the present disclosure, and

FIG. 6 is a schematic diagram of using multiple image selected boxes of different sizes to mark the tumor in the slice image information corresponding to the selected angle of the slice according to an embodiment of the present disclosure.

### DETAILED DESCRIPTION

Reference will now be made in detail to the present embodiments of the disclosure, examples of which are illustrated in the accompanying drawings. Wherever possible, the same reference numbers are used in the drawings and the description to refer to the same or like parts.

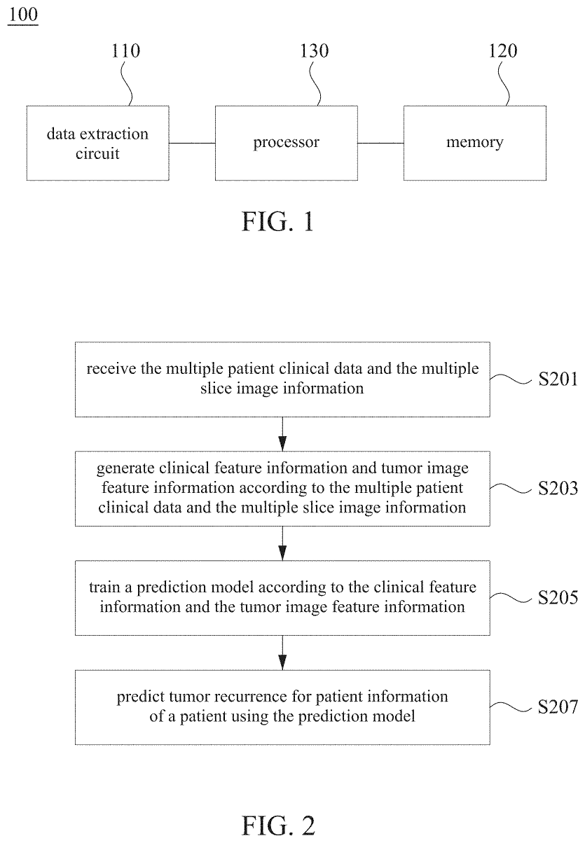

FIG. 1 is a block diagram of a tumor recurrence prediction device according to an embodiment of the present disclosure. Referring to FIG. 1, the tumor recurrence prediction device 100 includes a data extraction circuit 110, a memory 120 and a processor 130. The data extraction circuit 110 can extract various clinical data related to multiple patients who have previously accepted and finished a tumor treatment, and extract the slice image information corresponding to multiple angles of slices of the respective tumors of multiple patients who have previously accepted and finished the tumor treatment (each patient can have one or more tumors before receiving the tumor treatment).

In some embodiments, the tumor recurrence prediction device 100 is, for example, an electronic device such as a smart phone, a tablet computer, a notebook computer, a desktop computer etc., and other electronic devices that can connect to the Internet.

In some embodiments, the data extraction circuit 110 includes a circuit for acquiring magnetic resonance imaging (MRI) images and a circuit for acquiring multiple clinical data of multiple patients, where the circuit used to obtain MRI images is, for example, a circuit that uses MRI technology to scan patients and obtain the MRI images.

However, in other embodiments, the data extraction circuit 110 can also be configured to receive the MRI images and multiple clinical data of multiple patients from the memory 120 of the tumor recurrence prediction device 100 or other external storage devices. In addition, in other embodiments, the data extraction circuit 110 can also obtain the above-mentioned MRI images and multiple clinical data of multiple patients by other methods.

It is worth noting that the present disclosure is not used to limit the way the data extraction circuit 110 obtains the MRI images and the multiple clinical data of the multiple patients.

In some embodiments, the register 120 is, for example, any type of fixed or removable random access memory (RAM), read-only memory (ROM), flash memory (flash memory), hard disk drive (HDD), solid state drive (SSD) or similar components or a combination of the above components.

In some embodiments, the processor 130 is, for example, a central processing unit (CPU), or other programmable general-purpose or special-purpose micro control unit (MCU), microprocessor, digital signal processor (DSP), programmable controller, application specific integrated circuit (ASIC), graphics processing unit (GPU), arithmetic logic unit (ALU), complex programmable logic device (CPLD), field programmable gate array (FPGA) or other similar components or combinations of the above components.

FIG. 2 is a flowchart of a tumor recurrence prediction method according to an embodiment of the present disclosure. Referring to FIG. 1 and FIG. 2 at the same time, the method of this embodiment is applicable to the tumor recurrence prediction device 100 of FIG. 1. The following describes the detailed steps of the tumor recurrence prediction method according to the embodiment of the present disclosure in conjunction with the operation relationship between the devices in the tumor recurrence prediction device 100.

First, in step S201, the processor 130 can receive the multiple patient clinical data and the multiple slice image information. In detail, after the data extraction circuit 110 extracts the multiple patient clinical data and multiple slice image information of multiple patients who have previously accepted and finished the tumor treatment, the processor 130 can receive multiple patient clinical data and multiple slice image information from the data extraction circuit 110.

In some embodiments, the multiple patient clinical data include data of multiple clinical variables of multiple patients, such as age, gender, epidermal growth factor receptor (EGFR), whole brain radiotherapy (WBRT), using tyrosine kinase inhibitors (TKI) before, using TKI after, Karnofsky performance score (KPS), tumor recurrence, tumor number and tumor volume etc., and other various types of patient clinical data.

In some embodiments, the multiple slice image information is image information of multiple angles of the slices of multiple tumors corresponding to multiple patients, and the image information of each angle of the slice includes image information of multiple image types of the slice, such as tumor T1 weighted images (T1WI), T2 weighted images (T2WI) and contrast-enhanced T1 weighted images (T1WI+C) etc. and other types of MRI image information.

Next, in step S203, the processor 130 can generate clinical feature information and tumor image feature information according to the multiple patient clinical data and the multiple slice image information. In detail, the processor 130 can perform feature extraction on the multiple patient clinical data to generate the clinical feature information, and perform feature extraction on the multiple slice image information to generate the tumor image feature information.

In some embodiments, the processor 130 can generate a clinical data matrix based on the multiple patient clinical data, and generate multiple tumor image arrays based on the multiple slice image information. In this way, the processor 130 can perform feature extraction using the clinical data matrix directly to generate the clinical feature information, and perform feature extraction using the multiple slice image arrays directly to generate the tumor image feature information, where the clinical feature information is a clinical feature vector, and the tumor image feature information is a tumor image feature vector.

In further embodiments, the processor 130 can identify multiple tumor location information in the multiple slice image information to generate multiple tumor image information so as to generate multiple tumor image arrays based on the multiple tumor image information. In detail, the processor 130 can identify the tumor location information in each slice image from the multiple slice image information, and mark the tumor image information corresponding to the tumor location information using multiple image selected boxes of different sizes, where the image size of the each slice image can be any size, and there is no particular limitation. In addition, the method for identifying the multiple tumor location information can be any algorithm related to artificial intelligence or deep learning, and there is no particular limitation.

In further embodiments, by using the above-mentioned preprocessing method for generating the clinical data matrix and the multiple tumor image arrays, the processor 130 can further generate the clinical feature information using deep survival networks according to the clinical data matrix, and generate the tumor image information using image feature extraction networks according to the multiple tumor image information. In addition, the above-mentioned image feature extraction networks are, for example, spatial pyramid pooling networks (SPP-net) or pretrained deep learning model etc.

Next, in step S205, the processor 130 can train a prediction model according to the clinical feature information and the tumor image feature information. In detail, the processor 130 can use the clinical feature information and the tumor image feature information as multiple training samples, and train the prediction model using these training samples.

In some embodiments, the processor 130 can combine the clinical feature information and the tumor image feature information to generate a feature array, and train the prediction model using another deep survival networks according to the feature array.

Finally, the processor 130 can predict tumor recurrence for patient information of a patient using the prediction model. In detail, after completing the above-mentioned training phase, the processor 130 can further receive patient information of a patient who also has previously accepted and finished the tumor treatment from the memory 120 or an external storage device, and perform the tumor recurrence prediction according to the patient information. In this way, the processor 130 can determine whether the tumor of the tested patient recurs in the future, and identify a recurrence time in the condition of the tumor being possibly to recur (e.g. it will recur five years after finishing the tumor treatment). By the above determining and identifying results, the doctor can further continuously track the patient's physical condition after the patient has previously accepted and finished the tumor treatment, so as to predict the patient's tumor recurrence.

In some embodiments, the patient information of the above-mentioned patient includes clinical data such as age, gender, EGFR, WBRT, TKI before, TKI after, KPS, number of tumors, and tumor volume of the patient etc.

Based on the above, the tumor recurrence prediction device 100 of the present disclosure can not only predict tumor recurrence for patients who have previously accepted and finished the tumor treatment, but also predict the recurrence time for patient whose tumor is possibly to recur. In addition, the tumor recurrence prediction device 100 of the present disclosure also performs preprocessing and feature extraction on the multiple patient clinical data and the multiple slice image information at the same time to train a prediction model. In this way, the prediction accuracy of the prediction model can be effectively improved, thereby greatly reducing the occurrence of prediction errors.

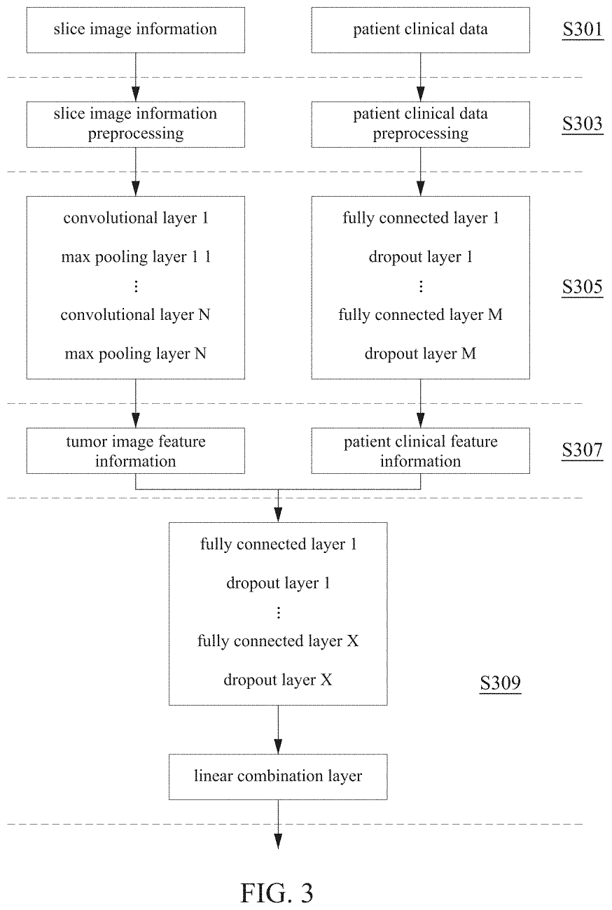

FIG. 3 is a schematic diagram of the tumor recurrence prediction method according to an embodiment of the present disclosure. Compared with the embodiment in FIG. 2, FIG. 3 discloses a more specific embodiment. Referring to FIG. 1 and FIG. 3 at the same time, first, in step S301, the processor 130 can retrieve the multiple patient clinical data and the multiple slice image information through the data extraction circuit 110. In detail, the processor 130 can extract clinical data of multiple patient clinical data types corresponding to multiple patients through the data extraction circuit 110, and extract image information of multiple slice image information types corresponding to multiple tumors corresponding to multiple patients.

For example, the processor 130 can extract the patient clinical data corresponding to the first patient among the multiple patients through the data extraction circuit 110, and the patient clinical data includes age, gender, EGFR, WBRT, TKI before, TKI after, KPS, whether the tumor recurred, the number of tumors, and the tumor volume of the first patient. By analogy, the processor 130 can extract the above-mentioned patient clinical data of the remaining patients among the multiple patients through the data extraction circuit 110. In addition, when the first patient has treated two tumors through the tumor treatment and photographed T1, T2, and T1c weighted images of two angles of the slices of the two tumors, the processor 130 can extract the T1, T2, and T1c weighted images (i.e. six images) of two angles of the slices of two tumors through the data extraction circuit 110 to generate its own slice image information of each tumor. By analogy, the processor 130 can extract the above-mentioned multiple slice image information of multiple tumors corresponding to the remaining patients of the multiple patients through the data extraction circuit 110.

Next, in step S303, the processor 130 can preprocess the clinical data of the multiple patients and the image information of the multiple slices to generate the clinical data matrix and the multiple tumor image arrays. In detail, in order to perform the feature extraction on the multiple patient clinical data and the multiple slice image information, the processor 130 needs to preprocess the multiple patient clinical data and the multiple slice image information.

In some embodiments, the processor 130 can perform right-censored processing on the clinical data of the multiple patients to generate the clinical data matrix. In detail, the processor 130 can identify that the multiple tumors correspond respectively to which patient clinical data according to the multiple patient clinical data, and generate the clinical data matrix according to the patient clinical data corresponding to the each tumor, where the multiple tumors correspond to multiple rows of the clinical data matrix, and multiple patient clinical data types corresponding to the multiple patient clinical data correspond to multiple columns of the clinical data matrix.

For example, for the first patient among the multiple patients, the processor 130 can extract age, gender, EGFR, WBRT, TKI before, TKI after, KPS, whether the tumor recurred, the number of tumors, and the tumor volume of the first patient, and identify that the first patient had treated two tumors according to the number of tumors in the first patient. In this way, the processor 130 can map the first tumor and the second tumor to the first row and the second row of the clinical data matrix, and map the patient clinical data types such as age, gender, EGFR, WBRT, TKI before, TKI after, KPS, whether the tumor recurred, the number of tumors, and the tumor volume of the first patient etc. to the columns of the clinical data matrix. Accordingly, the processor 130 can simultaneously indicate the data in the first row and the second row of the clinical data matrix as age, gender, EGFR, WBRT, TKI before, TKI after, KPS, whether the tumor recurred, and the tumor volume of the first patient.

By the same method, the processor 130 can identify age, gender, EGFR, WBRT, TKI before, TKI after, KPS, whether the tumor recurred, and tumor volume of the patients corresponding to the remaining tumors to generate a clinical data matrix.

In some embodiments, the processor 130 can perform image alignment processing, skull removal processing, and averaging processing on the multiple slice image information corresponding to the multiple tumors, where the averaging processing is, for example, various averaging processing such as Z score normalization processing of image gray scale intensity.

Further, the processor 130 can identify the slice image information corresponding to the each tumor, where the slice image information includes T1, T2, and T1c weighted images photographed from the multiple angles. In this way, the processor 130 can perform image alignment processing, skull removal processing, and averaging processing on the T1, T2, and T1c weighted images photographed from various angles.

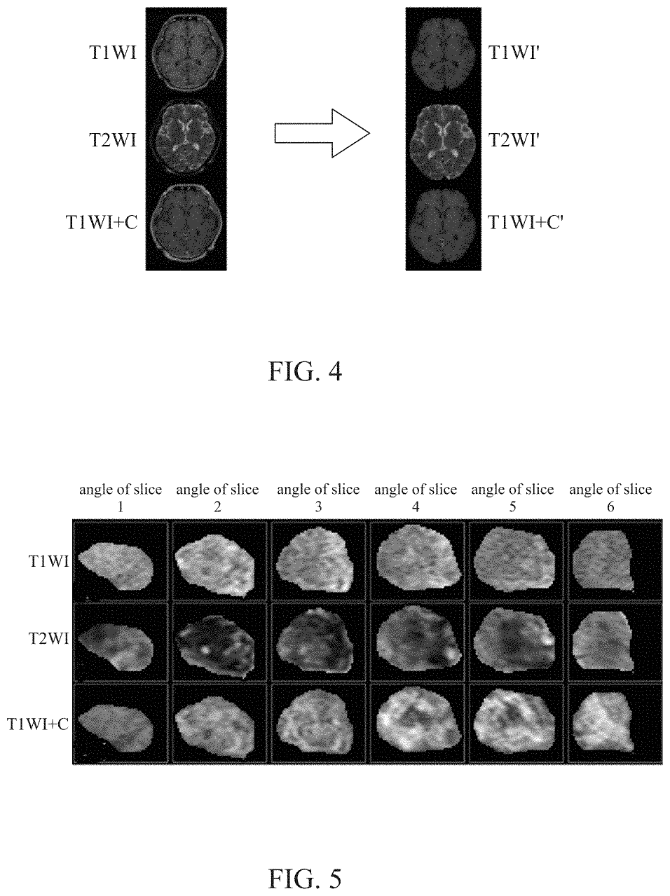

For example, FIG. 4 is a schematic diagram of performing image processing on slice image information according to an embodiment of the present disclosure. Referring to FIG. 4, the slice image information of one tumor of one patient includes T1WI, T2WI, and T1WI+C. First, the T1WI, the T2WI, and the T1WI+C can be aligned through the image alignment process, and can generate the T1WI′, the T2WI′, and the T1WI+C′ by the skull removal processing and the Z score normalization processing of the image gray scale intensity.

Referring back to FIGS. 1 and 3 at the same time, in a further embodiment, the processor 130 can identify the multiple tumor location information among the multiple processed slice image information, and generate multiple tumor image arrays according to the multiple tumor location information. Further, by the above-mentioned image alignment processing, the above-mentioned skull removal processing and the above-mentioned averaging processing, the processor 130 can identify the multiple tumor position information among the multiple slice image information of the multiple angles of the slices corresponding to each tumor, and detect the tumor size of the multiple slice image information according to the multiple tumor location information, so as to select the angle of the slice corresponding to the largest tumor size among the multiple angles of the slices. In addition, the processor 130 can mark the tumor in the slice image information corresponding to the selected angle of the slice using the multiple image selected boxes of different sizes to generate the tumor image information corresponding to the multiple image selected boxes. In this way, the processor 130 can generate the tumor image array of the each tumor according to the tumor image information corresponding to the each tumor.

For example, FIG. 5 is a schematic diagram of a tumor image corresponding to tumor location information according to an embodiment of the present disclosure. Referring to FIG. 5, for one tumor, six of slice image information can be photographed from angles 1 to 6 of the slices, where each of slice image information includes T1, T2, and T1c weighted images. In this way, the tumor location information in the T1, T2, and T1c weighted images of each of slice image information can be identified, the tumor images can be identified according to the tumor location information, and the tumor sizes can be identified according to the tumors image. Furthermore, since the tumor corresponding to the T1, T2, and T1c weighted images of the angle 4 of the slice has the largest tumor size, the slice image information corresponding to the angle 4 of the slice can be selected for subsequent image selection actions.

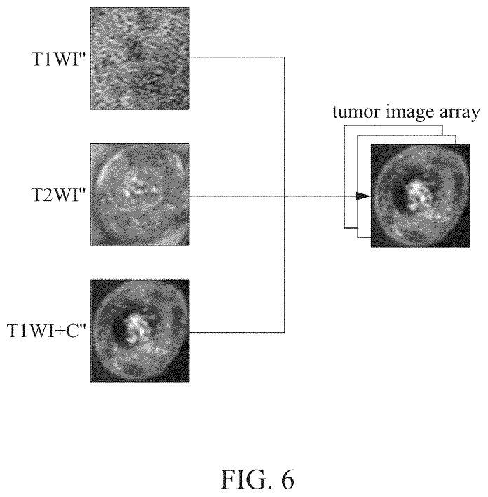

FIG. 6 is a schematic diagram of using multiple image selected boxes of different sizes to mark the tumor in the slice image information corresponding to the selected angle of the slice according to an embodiment of the present disclosure. Referring to FIG. 6, taking the image selected box with a size of 64×64 pixels as an example, this image selected box can be used to select the tumors in the T1, T2, and T1c weighted images in the slice image information to generate tumor image information including T1WI″, T2WI″ and T1WI+C″, where the sizes of the T1WI″, T2WI″ and T1WI+C″ are 64×64×1 pixels. In this way, the T1WI″, T2WI″ and T1WI+C″ in the tumor image information of this tumor can be overlapped to generate the tumor image array with a size of 64×64×3 pixels.

Next, referring back to FIGS. 1 and 3 at the same time, in step S305, the processor 130 can perform the feature extraction according to the clinical data matrix and the multiple tumor image arrays to generate the clinical feature information and the tumor image feature information. In detail, in order to generate the prediction model for the tumor recurrence prediction, the processor 130 needs to further perform the feature extraction on the clinical data matrix and the multiple tumor image arrays.

In some embodiments, the processor 130 can to generate the clinical feature information from the clinical data matrix using fully-connected layers 1˜M and dropout layers 1˜M in the deep survival networks, where the clinical feature information is a clinical feature vector. In addition, the processor 130 can generate the tumor image feature information from the multiple tumor image arrays using convolutional layers 1˜N, max-pooling layers 1˜N, and spatial pyramid pooling layers in SPP-net, where the tumor image feature information is a tumor image feature vector. It is worth noting that M and N are the best positive integers tested through many experiments.

Next, in step S307, the processor 130 can combine the clinical feature information with the tumor image feature information.

In some embodiments, the clinical feature information is a clinical feature vector, and the tumor image feature information is a tumor image feature vector.

Finally, in step S309, the processor 130 can train the prediction model using the combined clinical feature information and tumor image feature information. In detail, the processor 130 can use the clinical feature information and the tumor image feature information as the training samples to train the prediction model.

In some embodiments, the processor 130 can combine the clinical feature information with the tumor image feature information to generate a feature vector, and generate the prediction model using fully-connected layers 1˜X, dropout layers 1˜X and a linear combination layer in the deep survival networks according to the feature vector. It is worth noting that X is also the best positive integer tested through many experiments.

In summary, the tumor recurrence prediction device provided by the present disclosure combines the feature extraction of the multiple patient clinical data and the multiple tumor image information, and train the prediction model using the extracted feature information to solve the problem that the accuracy of the current survival prediction analysis is not great enough. In this way, the prediction model provided by the present disclosure more accurately predict whether the tumor of the patient, who has previously accepted and finished the tumor treatment, will recur and the time for recurrence.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A tumor recurrence prediction device, comprising:
a data extraction circuit configured to extract a plurality of patient clinical data and a plurality of slice image information;
a memory configured to store a plurality of instructions;
a processor connected to the data extraction circuit and the memory, and configured to load and execute the plurality of instructions to:
receive the plurality of patient clinical data and the plurality of slice image information;
generate clinical feature information and tumor image feature information according to the plurality of patient clinical data and the plurality of slice image information;
train a prediction model according to the clinical feature information and the tumor image feature information; and
predict tumor recurrence for patient information of a patient using the prediction model.

2. The tumor recurrence prediction device of claim 1, wherein the processor is further configured to:
generate a clinical data matrix according to the plurality of patient clinical data, and generate a plurality of tumor image arrays according to the plurality of slice image information.

3. The tumor recurrence prediction device of claim 2, wherein the processor is further configured to:
identify a plurality of tumor position information in the plurality of slice image information; and
generate a plurality of tumor image information according to the plurality of tumor position information, and generate the plurality of tumor image arrays according to the plurality of tumor image information.

4. The tumor recurrence prediction device of claim 2, wherein the processor is further configured to:
generate the clinical feature information using deep survival networks according to the clinical data matrix; and
generate the tumor image feature information using image feature extraction networks according to the plurality of tumor image arrays.

5. The tumor recurrence prediction device of claim 2, wherein the processor is further configured to:
combine the clinical feature information with the tumor image feature information to generate a feature array, and train the prediction model using deep survival networks according to the feature array.

6. A tumor recurrence prediction method, comprising:
generating patient feature information and tumor image feature information according to a plurality of patient clinical data and a plurality of slice image information;
combining the clinical feature information with the tumor image feature information to generate a feature array, and training a prediction model according to the feature array; and
predicting tumor recurrence for patient information of a patient using the prediction model.

7. The tumor recurrence prediction method of claim 6, wherein the step of generating the patient feature information and the tumor image feature information according to the plurality of patient clinical data and the plurality of slice image information comprises:
generating a clinical data matrix according to the plurality of patient clinical data, and generating a plurality of tumor image arrays according to the plurality of slice image information.

8. The tumor recurrence prediction method of claim 7, wherein the step of generating the plurality of tumor image arrays according to the plurality of slice image information comprises:
identifying a plurality of tumor position information in the plurality of slice image information; and
generating a plurality of tumor image information according to the plurality of tumor position information, and generate the plurality of tumor image arrays according to the plurality of tumor image information.

9. The tumor recurrence prediction method of claim 7, further comprising:
generating the clinical feature information using deep survival networks according to the clinical data matrix; and
generating the tumor image feature information using image feature extraction networks according to the plurality of tumor image arrays.

10. The tumor recurrence prediction method of claim 6, wherein the step of training the prediction model according to the feature array comprises:
training the prediction model using deep survival networks according to the feature array.
